(19) United States
(12) Patent Application Publication
(10) Pub. No.: US 2017/0260562 A1
(43) Pub. Date: Sep. 14, 2017

(54) PETRI DISH AND METHOD FOR THE MICROBIOLOGICAL EXAMINATION OF LIQUIDS BY MEMBRANE FILTRATION

(71) Applicant: MERCK PATENT GMBH, Darmstadt (DE)

(72) Inventors: Reiner HEDDERICH, Hirschberg (DE); Anne-Grit KLEES, Darmstadt (DE); Andreas BUBERT, Gross-Bieberau (DE)

(73) Assignee: Merck Patent GmbH, Darmstadt (DE)

(21) Appl. No.: 15/503,641

(22) PCT Filed: Jul. 17, 2015
(86) PCT No.: PCT/EP2015/001497

§ 371 Date: Feb. 13, 2017

(30) Foreign Application Priority Data
Aug. 14, 2014 (EP) 14002840.8

(51) Int. Cl. C12Q 1/04; C12M 1/00; C12M 3/00; C12M 1/22

(52) U.S. Cl. CPC C12Q 1/04; C12M 23/10; C12M 23/38; C12M 23/48

(57) ABSTRACT

The invention relates to a medium-filled Petri dish into which filters can be introduced particularly simply, and to a method for the microbiological investigation of liquids or gases by means of filtration and incubation of the filters in said Petri dishes.

The invention relates to a medium-filled Petri dish into which filters can be introduced particularly simply, and to a method for the microbiological investigation of, in particular, liquids by means of filtration and incubation of the filters in said Petri dishes.

Bacteria, moulds and yeasts can negatively impair the quality and keeping properties of liquids, such as, for example, drinks, water or cosmetics. Microbes of this type can be found in the raw materials, in the air or also on surfaces during processing.

It is therefore typically attempted even during production on the one hand to prevent the contamination and on the other hand to discover possible contamination by regular sampling and testing at key points. A systematically carried out programme of sampling and analysis enables the manufacturer to discover and eliminate contamination at the source before it develops into a greater problem.

The microbiological analysis of liquids to be filtered is typically carried out using filters, through which the liquids to be investigated flow. After filtration, the filter is removed and placed, for example, in agar dishes, on which the filter is stored at elevated temperature in an incubator for several days. The agar provides any microbes which have been filtered out of the liquid with nutrients, which stimulate growth, so that the microbes are able to reproduce and can be determined or counted.

A principal difficulty of this known method is the handling of the filters, which are usually extremely sensitive. They are typically gripped using tweezers and positioned on the medium of the Petri dish. The filter and the medium of the Petri dish must not be damaged or deformed during the handling.

A further problem, in particular for laboratories in which a large number of samples have to be investigated in parallel, is the space requirement of the individual Petri dish. In general, use is made of Petri dishes which have a significantly greater diameter than the filter to be investigated. This is necessary since otherwise the filter cannot be placed on the medium, which is located well below the rim of the dish. For example, Petri dishes having a diameter of 60 mm or more are used for filters having a diameter of 47 mm. The use of large Petri dishes means greater consumption of materials for the Petri dish and the medium located therein and in particular a greater space requirement in the investigating laboratory during incubation of the dishes.

The object of the present invention was therefore to simplify handling of the filters during placing on the Petri dish and in addition to provide a dish which saves as much space as possible.

It has been found that the method, which has been known for decades and has been carried out using many, in some cases complicated devices can be considerably simplified by using a Petri dish which is filled with medium virtually completely or preferably up to the rim. In this way, the filter can be placed and positioned on the medium without hindrance by the rim of the Petri dish. Handling is faster and with less damage to the filter or medium. Furthermore, space and medium can thereby be saved, since the diameter of the Petri dish can be reduced to virtually the diameter of the filter.

The present invention therefore relates to a Petri dish comprising a dish, which is filled with medium at least to 1 mm below the rim of the dish, but at most to the rim of the dish, and a lid, which closes the dish.

In a preferred embodiment, the dish is filled with medium completely, i.e. to the rim.

In another preferred embodiment, the medium is an agar medium.

In another embodiment, the dish and lid are round.

In a preferred embodiment, the lid has an at least slightly greater inside diameter than the outside diameter of the dish, so that the lid can be inverted over the dish in order to close the dish.

In another preferred embodiment, the base of the lid does not rest on the rim of the dish. To this end, one or more devices which have the effect that the base of the lid does not rest directly on the rim of the dish are preferably located on the wall of the dish or on the lid.

In an embodiment, the resting of the base of the lid is prevented by at least 3 protruberances, on which the lid rests, being installed on the rim of the dish.

In another embodiment, holders, for example in the form of a bead or in the form of sealing devices, on which the rim of the lid rests, are mounted on the wall of the dish (inside or preferably outside).

The present invention also relates to a method for the determination of microbes in liquids or gases, characterised by the following method steps
a) filtration of the liquid or gas through a filter
b) application of the filter to the medium of a Petri dish according to the invention
c) sealing of the Petri dish by means of the lid
d) incubation of the Petri dish from step c)
e) evaluation of the microbe growth

In a preferred embodiment, a liquid is filtered.

In a preferred embodiment, filter and Petri dish are round shaped.

In a preferred embodiment, the internal diameter of the Petri dish is 2 to 10 mm greater than the diameter of the filter.

The filter preferably has an exclusion limit between 0.2 and 0.45 μm.

The filter preferably consists predominantly of cellulose acetate and/or cellulose nitrate.

The present invention also relates to the use of a Petri dish according to the invention for the determination of microbes on filters. To this end, the filters are placed on the medium of the Petri dish, incubated and subsequently the microbes are evaluated.

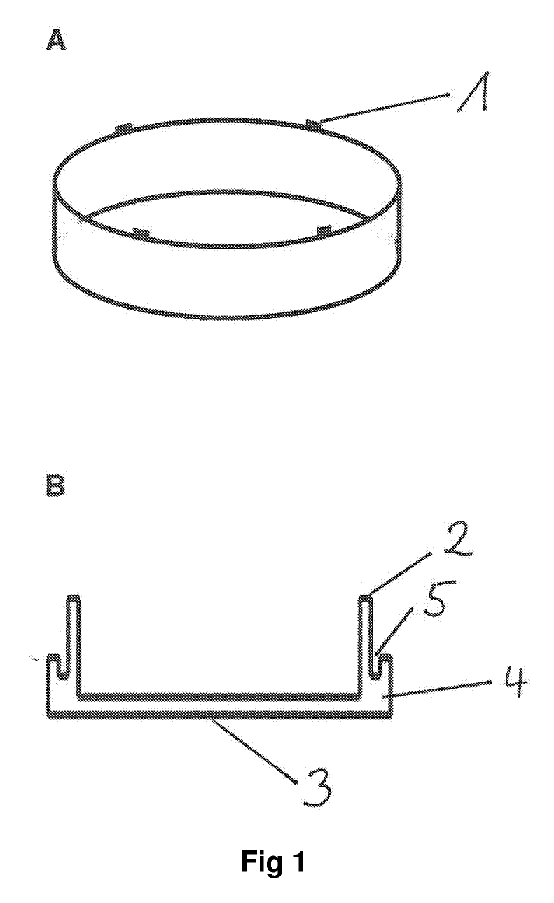

FIG. 1 shows two possible embodiments of the dish of a Petri dish according to the invention. The lid is not shown in either case.

In accordance with the invention, a Petri dish is a container for the accommodation of nutrient media, which are taken to mean, for example, nutrient solutions or solid culture media, in particular for the cultivation of microorganisms, cell cultures, bacteria, etc. The container comprises a dish and a lid which seals the dish. It should be pointed out at this point that the lid may engage around the dish or engage into the dish. Consequently, the dish and lid should very generally be taken to mean parts which engage with one another to form a more or less sealed-off space. For simplification, the terms dish and lid will be used hereinafter.

The dish and lid each comprise a base (dish base and lid base), preferably in the form of a circular area, and a wall (dish wall and lid wall) projecting from the base, preferably in the form of a circular ring. The upper edge of the wall is called the rim. One of the walls, preferably the lid wall, has an at least slightly greater inside diameter than the outside diameter of the other wall, preferably the dish wall, so that, in order to close the dish, one wall can be inverted over the other (or vice versa).

A Petri dish is thus typically a flat, round, usually transparent glass or plastic dish with preferably overlapping lid. A dish of this type is usually used in biology or chemistry. It serves for the cultivation of microorganisms, also called microbes, and is utilised to establish cell cultures.

In relation to the generic container, reference may be made merely by way of example to DE 44 06 725 A1, from which a container in the sense of a Petri dish is known. A lid serves to seal the container. The lid is generally inverted over the dish or over the dish wall.

Media in the sense of the present invention are solid nutrient media or culture media, on which various microbes can grow specifically or certain microbes can grow specifically. The basic composition of a medium usually consists of the principal component water, an energy source which can be utilised for the respective microbe, for example organic compounds, and nutrients required by it (organic or inorganic carbon, nitrogen, sulfur and phosphate sources and other essential nutrients). The nutrients in the nutrient media for heterotrophs are usually carbohydrates (“sugars”), protein hydrolysates (peptones) and optionally fatty acids. In addition, inorganic salts provide the microbes with essential ions, such as, for example, ammonium, potassium, sodium, phosphate, sulfate and trace elements. In addition, the following may also be present:
dyes or precursors thereof (microscopy dyes, chromogenic substrates)
gelling agents (solidifiers), such as agar or gelatin,
inhibitors (for example antibiotics) and selective agents for preventing the growth of undesired microorganisms (for example chloramphenicol for yeast/mould culture media)
indicators for indicating changes, such as, for example, in the pH, but also in order to indicate certain metabolic products or metabolic activities
buffer substances in order to stabilise the pH
growth factors, such as hormones, vitamins and the like.

Preference is given in accordance with the invention to agar-based media, i.e. media which comprise agar.

Media for the determination of microbes are known to the person skilled in the art. Examples of suitable media are found, for example, in Microbiology Manual 2000, Merck KGaA, Germany.

The following table shows by way of example some media and the microbes to be determined with them.

| Medium | Microbe |
|---|---|
| m-Green | Yeasts and mould fungi |
| mTGE | All (total count) |
| Cetrimide Nalidixic acid | Pseudomonas aeruginosa |
| Slanetz Bartley | Enterococci |
| Chromocult ® coliform | E. coli/coliforms |
| Lactose TTC | E. coli/coliforms |
| m-Endo | Coliforms |
| mFC | Coliforms |
| TSC | Clostridia perfringens |
| mCP | Clostridia perfringens |
| BCYE | Legionella spp. |
| GVPC | Legionella spp. |
| BAT | Alicyclobacillus |

Filters in the sense of the present invention are all filters which are suitable for the collection of microbes, such as mould fungi, yeasts or bacteria.

These can be paper filters or membrane filters, for example made from plastic. For example, filters made from PVDF are suitable. Particular preference is given to filters based on cellulose mixed esters. These are filters which comprise at least cellulose acetate and/or cellulose nitrate. The pore size of the filters depends on the size of the microbes to be isolated. Typical exclusion sizes are 0.2 to 0.45 μm. This means that the filters have pore sizes between 0.2 to 0.45 μm.

Use is typically made of filters having diameters between 30 and 80 mm, preferably between 40 and 50 mm.

An illustrative filter which is suitable for use in the method according to the invention is EZ-PAK® Membrane, from Merck Millipore, Germany. This is a membrane filter based on cellulose esters having a pore size of 0.45 μm.

It has been found that a Petri dish according to the invention which is filled with medium at least to 1 mm below the rim of the dish, but at most to the rim of the dish, significantly simplifies analysis of filters. On the one hand, it is significantly simpler to position the sensitive filters on the medium of the dish. On the other hand, the diameter of the dish can be matched to that of the filter. Particularly in laboratories in which a large number of samples have to be investigated in parallel, the space taken by the individual Petri dish should be as small as possible. The present invention now offers the possibility of employing Petri dishes which are smaller in relation to the diameter of the filter, since the placing of the filter on a Petri dish filled to the rim is significantly simpler than placing the filter deep down in the dish.

The dish is typically filled with medium to at least 1 mm below the rim of the dish, but at most to the rim of the dish. The dish is preferably filled with medium precisely to the rim, i.e. completely.

The height of the media filling arises from the wall height of the Petri dish. Wall height here means the wall height of the inside of the Petri dish. This differs from the outside wall height of the dish by the thickness of the base of the Petri dish. The inside wall height of the Petri dish should be at least 3 mm, so that a height of the medium of at least 3 mm arises. The height of the medium is preferably between 3 and 7 mm. Preferred wall heights of 3 to 8 mm thus arise for the Petri dish. The choice of the wall height of the Petri dish enables the consumption of medium to be influenced.

The medium is preferably an agar medium.

In a preferred embodiment, the dish and lid are round and the lid has an at least slightly greater inside diameter than the outside diameter of the dish, so that the lid can be inverted over the dish in order to close the dish.

In order to facilitate aerobic incubation, which is typically desired, and to prevent contact between filter and lid, dish and lid are preferably designed in such a way that the lid base does not rest directly on the rim of the dish. This can be achieved in various ways. For example, the rim of the dish can have at least 3 protuberances on which the lid rests. The lid consequently rests in a stable manner, but, due to the protuberances, has a separation from the actual rim of the dish. An embodiment of this type is shown diagrammatically in FIG. 1A. The figure shows the round dish, on the rim of which four protuberances are at the top. One of the four protuberances is marked with the number “1” in the figure for better understanding. If a lid is now placed on such a dish, the protuberances generate a separation between the lid base and the rim of the dish.

In a further embodiment, the wall of the dish can have devices, such as an inside or outside bead or one or more holders, on which the rim of the lid rests. If the height of the lid wall is now higher than the height of the wall of the dish above the bead or the holder, the base of the lid does not rest on the rim of the dish.

In an embodiment, the bead is designed as an outside circular recess into which the lid engages. An embodiment of this type is depicted diagrammatically in FIG. 1B. The figure shows a section through the dish consisting of wall (2) and base (3). A bead (4), which runs around the entire wall in a ring-shaped manner, is located outside on the wall (2). The lid is able to engage in the recess or channel (5) thus formed. If the wall of the lid is now selected correspondingly high, the lid base does not rest on the rim of the dish.

In another embodiment, outside holders not only facilitate the separation between lid base and dish rim, but additionally also effect locking of the lid. For this purpose, the holders can be designed, for example, as outwardly protruding snap-in lugs, flanges, thread-like parts, combinations of different engagement means or the like. Sealable Petri dishes of this type are known, for example, from WO 2008/141597.

The present invention also relates to a method for the determination of microbes and liquids or gases, characterised by the following method steps
a) filtration of the liquid or gas through a filter
b) application of the filter to the medium of a Petri dish according to the invention
c) sealing of the Petri dish by means of the lid
d) incubation of the sealed Petri dish from step c)
e) evaluation of the microbe growth

The general method for the investigation of liquid or gaseous samples by filtration and subsequent incubation of the filter in a Petri dish is known to the person skilled in the art. Liquids here are, in particular, water, drinks, liquid foods or liquid cosmetic articles. The most investigated gas is air, for example in pharmaceuticals or drinks and/or food production.

The method is typically carried out as follows.

### 1. Preparation of Filter and Petri Dish

In accordance with the invention, a Petri dish is provided which is filled with medium at least to 1 mm below the rim, at most to the rim.

In a preferred embodiment, filter and Petri dish are round shaped.

In a preferred embodiment, the diameter in the interior of the Petri dish is 2 to 10 mm, preferably 1 to 8 mm greater than the diameter of the filter. In this way, it is possible to use a Petri dish having the smallest possible diameter. For filters having a diameter of 47 mm, Petri dishes having a diameter of 55 mm, for example, are thus suitable.

Further necessary and preferred properties of Petri dish and filter have already been disclosed above.

2. The Filter is Introduced into a Funnel or Another Device for the Filtration of Liquids or Gases.

Devices of this type are known to the person skilled in the art. Examples of devices for the filtration of liquids are found in WO2008113443 and the documents cited therein.

Typically, between 50 and 500 ml, preferably between 100 and 250 ml of the liquid are passed through the filter. The microbes remain adhering to the filter.

3. Removal of the Filter from the Filtration Device.

The filter can optionally be rinsed before removal from the filtration device in order to exclude possible interfering influences of the original liquid during subsequent determination of the microbes.

4. Introduction of the Filter into the Petri Dish

The filter is typically placed on the medium of the opened Petri dish using sterile tweezers. The dish is then sealed by means of the lid.

### 5. The Sealed Petri Dish is Incubated

To this end, the Petri dish is typically placed in an incubator. Incubation time and incubation temperature are different for different microbe types. The incubation times are typically between 12 and 48 hours. The temperatures are typically about 36° C. For E. coli/coliforms on a chromogenic coliform agar, the incubation times are, for example, preferably between 18 and 24 hours at about 36° C. The person skilled in the art is able to match the temperature and incubation times to the respective microbes to be determined.

### 6. Evaluation of the Microbe Growth

After incubation, the plates can be removed from the incubator and evaluated. This can be carried out visually or using suitable instruments. Methods for evaluation are known to the person skilled in the art. The evaluation of the microbe growth can be carried out by counting the microbe colonies formed and/or by evaluation of further features. Counting of the colonies formed on the agar is typically carried out. If, for example, 20 colonies are found on the agar, it is assumed that 20 microbes have been filtered out of the sample. Depending on the sample volume employed, the microbe count per volume unit can be calculated therefrom. The evaluation of further features includes determination of a colour change, for example owing to a change in the pH or metabolisation of a chromogenic substrate. Evaluation of further features can, however, also mean isolation of one or more microbes and identification thereof by means of further tests, such as, for example, PCR or antibody-based tests.

The present invention thus provides a significant improvement in the determination of microbes in liquids or gases by means of filtration. The simple increase in the media filling level in the Petri dishes significantly simplifies handling. In addition, the size of the Petri dishes in relation to the filter can be reduced.

The illustrative embodiments explained above along with figures serve merely for illustrative explanation of the claimed teaching, but does not restrict this to the illustrative embodiments. Even without further comments, it will be assumed that a person skilled in the art will be able to utilise the above description in the broadest scope.

The complete disclosure content of all applications, patents and publications cited above and below, in particular the corresponding application EP 14002840.8, filed on 14 Aug. 2014, is incorporated into this application by way of reference.

1. A Petri dish comprising a dish, which is filled with medium at least to 1 mm below the rim of the dish, but at most to the rim of the dish, and a lid.

2. Petri dish according to claim 1, characterised in that the dish is filled with medium to the rim.

3. Petri dish according to claim 1, characterised in that the medium comprises agar.

4. Petri dish according to claim 1, characterised in that the dish and lid are round.

5. Petri dish according to claim 1, characterised in that the lid has an at least slightly greater inside diameter than the outside diameter of the dish, so that the lid can be inverted over the dish in order to close the dish.

6. Petri dish according to claim 1, characterised in that the lid base does not rest directly on the rim of the dish.

7. Petri dish according to claim 1, characterised in that at least 3 protuberances, on which the lid base rests, are installed on the rim of the dish.

8. Petri dish according to claim 1, characterised in that at least one holder for the lid is mounted outside on the wall of the dish.

9. A method for the determination of microbes in liquids or gases, characterised by the following method steps
a) filtration of the liquid or gas through a filter
b) application of the filter to the medium of a Petri dish according to claim 1
c) sealing of the Petri dish by means of the lid
d) incubation of the Petri dish from step c)
e) evaluation of the microbe growth

10. Method according to claim 9, characterised in that filter and Petri dish are round shaped.

11. Method according to claim 9, characterised in that the inside diameter of the Petri dish is 2 to 10 mm greater than the diameter of the filter.

12. Method according to claim 9, characterised in that the filter has an exclusion limit between 0.2 and 0.45 μm.

13. Method according to claim 9, characterised in that the filter consists of cellulose mixed esters.

14. (canceled)
